(19) United States
(12) Patent Application Publication
(10) Pub. No.: US 2016/0068565 A1
(43) Pub. Date: Mar. 10, 2016

(54) METHOD FOR HARVESTING CULTURE PRODUCT

(71) Applicant: ASAHI KASEI MEDICAL CO., LTD., Tokyo (JP)

(72) Inventors: Masafumi SHIBANO, Tokyo (JP); Chihiro KATO, Tokyo (JP)

(73) Assignee: ASAHI KASEI MEDICAL CO., LTD., Tokyo (JP)

(21) Appl. No.: 14/640,511

(22) Filed: Mar. 6, 2015

(30) Foreign Application Priority Data
Sep. 9, 2014 (JP) 2014-183641

(51) Int. Cl. C07K 1/34; B01D 63/02; C07K 16/00; B01D 71/58; B01D 71/68; B01D 61/14; B01D 69/02

(52) U.S. Cl. CPC C07K 1/34; B01D 61/145; B01D 63/02; B01D 69/02; B01D 71/58; B01D 71/68; C07K 16/00; B01D 2325/38

(57) ABSTRACT

The present invention provides a more productive method for culturing and a more productive method for harvesting culture product in cell culture wherein the cell produces the culture product. The present invention relates to a method for harvesting a culture product contained in a culture solution in the cell culture wherein the cell produces the culture product, comprising the following steps: B sending the culture solution to a filtration membrane; C: filtering the culture solution by alternating tangential flow filtration while changing the flow of the culture solution so as to cause a reciprocating motion thereof in a direction parallel with the surface of the filtration membrane to obtain a filtrate; D: sending back a culture solution residue that has remained without permeating the filtration membrane; and G: harvesting the culture product from the filtrate, wherein
the filtration membrane used in the step B is a porous membrane having an average pore size of 20 μm or larger and 100 μm or smaller for the pores in the surface of the culture solution side or a porous membrane in which the ratio of pores having a diameter smaller than 20 μm for the pores in the surface of the culture solution side is 50% or less of all pores in the surface of the culture solution side.

### BACKGROUND OF THE INVENTION

1. Field of the Invention

The present invention relates to a method for harvesting a culture product contained in a culture solution in the cell culture wherein the cell produces the culture product.

2. Background Art

Cell culture technology is a technique indispensable for the manufacture of various biotechnology-based drugs such as growth hormone and erythropoietin and has made a significant contribution to the recent advancement of medicine.

Industrial cell culture methods aimed at producing these useful substances are broadly divided into two types: adhesion culture and suspension culture (floating culture) methods. The suspension culture method is in the mainstream because of its easy scale-up, easy control at a large scale, etc.

For the methods for culturing cells by suspension culture, for example, culture methods utilizing a culture vessel such as a spinner flask equipped with an adjusted stirring function have been proposed, wherein a magnetic stirrer or an impeller on a mechanically driven shaft is employed as the stirring function. In these culture methods, however, the growth of cells is halted at a relatively low cell density because the cells are cultured in a given amount of nutrients. For such a suspension culture method of cells, methods for continuously maintaining the growing environment of cells in a culture vessel under the optimum conditions for a long period have been studied which involve efficiently separating a spent culture solution and a produced culture product from cells in a suspension over a long period and taking the spent culture solution and the culture product out of the culture vessel.

As means to carry out the aforementioned separation over a long period using a hollow fiber membrane, i.e., means to prevent the membrane from being contaminated due to usage, for example, Patent Literature 1 describes a method for inversing a culture solution flow between the supply of a fresh medium and the discharge of the culture solution. In recent years, use of a method called alternating tangential flow (ATF) filtration has permitted long-term high-density cell culture with reduced membrane contamination. This method is becoming useful in enhancing productivity.

Improved productivity leads to cost reduction for irreplaceable biotechnology-based drugs, expanded use of the drugs, and medical cost saving and thus has an immeasurable impact on the advancement of medicine.

Various devises or improvements have been made in the whole system of carrying out filtration in combination with culture. For example, Patent Literatures 2 to 4 disclose systems for achieving alternating tangential flow filtration. Also, Patent Literature 5 shows findings regarding the material, structure, pore size, and filtration pressure of a filtration membrane for carrying out filtration. Nonetheless, filtration membranes suitable for this purpose are still need to be improved.

### CITATION LIST

### Patent Literature

Patent Literature 1: Japanese Patent Laid-Open No. H02-200176
Patent Literature 2: National Publication of International Patent Application No. 2012-503540
Patent Literature 3: U.S. Pat. No. 6,544,424
Patent Literature 4: Japanese Patent No. 5003614
Patent Literature 5: Japanese Patent Laid-Open No. 2012-135249

### SUMMARY OF INVENTION

### Technical Problem

It has been revealed that, when the conventional filtration membranes for use in the alternating tangential flow filtration system are continuously used, the membrane permeability of a target substance (useful substance produced by cells) is reduced. This may not only result in the decreased recovery rate of the product but cause increase in product-derived impurities such as aggregates. The reduced recovery rate of the product leads to a rise in manufacture cost and a rise in medical cost. The increase in impurities such as aggregates may cause, for example, the production of neutralizing antibodies, which is responsible for side effect in patients who have used the product as a drug preparation. These are very serious problems associated with medicine.

Against such a background, a problem to be solved by the present invention is to provide a more productive method for culturing and a more productive method for harvesting culture product in cell culture wherein the cell produces the culture product and a method for harvesting the culture product. Another object of the present invention is to provide a filtration method using a filtration membrane suitable for alternating tangential flow filtration.

### Solution to Problem

The present inventors have conducted diligent studies to attain the objects and consequently found that, for the alternating tangential flow filtration of a culture solution of cells which produce a culture product, more productive culture can be achieved by using, in the alternating tangential flow filtration, a filtration membrane having no dense layer in a membrane surface of the culture solution side and a filtration membrane having an average pore size within a particular range for the pores in the surface of the culture solution side. Use of such a filtration membrane in the alternating tangential flow filtration for the culture of cells which produce a culture product can maintain the permeability of the product for a long period and reduce the formation of product-derived impurities. As a result, the cells which produce the culture product can be cultured with higher productivity.

Specifically, the present invention relates to the following methods:

[1]

A method for harvesting a culture product contained in a culture solution in cell culture wherein the cell produces the culture product, comprising following steps:

B: sending the culture solution to a filtration membrane;

C: filtering the culture solution by alternating tangential flow filtration while changing the flow of the culture solution so as to cause a reciprocating motion thereof in a direction parallel with the surface of the filtration membrane to obtain a filtrate;

D: sending back a culture solution residue that has remained without permeating the filtration membrane; and

G: harvesting the culture product from the filtrate, wherein

the filtration membrane used in the step B is a porous membrane having an average pore size of 20 μm or larger and 100 μm or smaller for the pores in the surface of the culture solution side.

[2]

A method for harvesting a culture product contained in a culture solution in cell culture wherein the cell produces the culture product, comprising the following steps:

B: sending the culture solution to a filtration membrane;

C: filtering the culture solution by alternating tangential flow filtration while changing the flow of the culture solution so as to cause a reciprocating motion thereof in a direction parallel with the surface of the filtration membrane to obtain a filtrate;

D: sending back a culture solution residue that has remained without permeating the filtration membrane; and

G: harvesting the culture product from the filtrate, wherein

the filtration membrane used in the step B is a porous membrane in which the ratio of pores having a diameter smaller than 20 μm for the pores in the surface of the culture solution side is 50% or less of all pores in the surface of the culture solution side.

[3]

The method for harvesting a culture product according to [1] or [2], wherein the filtration membrane is a porous membrane in which the average pore size of the pores in the surface of the culture solution side is larger than the average pore size of the pores in the surface of the filtrate side.

[4]

The method for harvesting a culture product according to any of [1] to [3], wherein the filtration membrane is a hollow fiber membrane.

[5]

The method for harvesting a culture product according to any of [1] to [4], wherein the filtration membrane has a minimum pore size of 0.1 μm or larger and 1 μm or smaller.

[6]

The method for harvesting a culture product according to any of [1] to [5], wherein the filtration membrane is a porous hollow fiber membrane constituted by a blend of a hydrophobic polymer and polyvinylpyrrolidone.

[7]

The method for harvesting a culture product according to [6], wherein the filtration membrane is a hollow fiber membrane in which, when the tube wall is equally divided into three areas in the membrane thickness direction, the content ratio of polyvinylpyrrolidone in an outer peripheral area including an external surface which is the surface of the filtrate side of the filtration membrane is larger than the content ratio of polyvinylpyrrolidone in an inner peripheral area including an internal surface which is the surface of the culture solution side of the filtration membrane.

[8]

The method for harvesting a culture product according to [6] or [7], wherein the hydrophobic polymer is polysulfone.

[9]

The method for harvesting a culture product according to any of [1] to [8], further comprising, prior to the step B,

A: culturing the cell which produces the culture product in the culture solution to produce the culture product.

[10]

The method for harvesting a culture product according to any of [1] to [9], further comprising, simultaneously with any of the steps B to D or prior to or after any of the steps B to D,

E: continuously and/or intermittently supplying a fresh culture solution.

[11]

The method for harvesting a culture product according to any of [1] to [10], further comprising, after the step D,

F: discharging the culture solution residue.

[12]

The method for harvesting a culture product according to any of [1] to [11], wherein the step A is carried out in the culture solution retained in a culture vessel.

[13]

The method for harvesting a culture product according to any of [1] to [12], wherein the step C is carried out using the filtration membrane housed in a cylindrical container.

[14]

The method for harvesting a culture product according to any of [1] to [13], wherein the filtration membrane permeation rate of the culture product 10 days after the start of the culture is 70% or more of the filtration membrane permeation rate of the culture product 3 days after the start of the culture.

[15]

The method for harvesting a culture product according to any of [1] to [14], wherein the ratio of aggregates in the culture solution 10 days after the start of the culture is less than 150% of the ratio of aggregates in the culture solution 2 days after the start of the culture.

[16]

The method for harvesting a culture product according to any of [1] to [15], wherein the ratio of aggregates in the filtrate 10 days after the start of the culture is less than 150% of the ratio of aggregates in the filtrate 2 days after the start of the culture.

[17]

The method for harvesting a culture product according to any of [1] to [16], wherein the culture product is a biologically active substance.

[18]

The method for harvesting a culture product according to any of [1] to [17], wherein the culture product is selected from the group consisting of proteins, viruses, exosomes, and nucleic acids.

[19]

The method for harvesting a culture product according to any of [1] to [18], wherein the culture product is an immunoglobulin.

[20]

The method for harvesting a culture product according to any of [1] to [19], wherein the culture is continuous culture.

[21]

The method for harvesting a culture product according to any of [1] to [20], wherein a glucose concentration in the culture solution is controlled at 1 g/L or higher and 15 g/L or lower.

[22]

The method for harvesting a culture product according to any of [1] to [21], wherein a lactic acid concentration in the culture solution is controlled at 0 g/L or higher and 2 g/L or lower.

[23]

The method for harvesting a culture product according to any of [1] to [22], wherein the protein concentration of the culture solution at which the membrane area-based cumulative throughput of the filtration membrane is 400 L/m² is twice or less the protein concentration of the culture solution at the start of the filtration.

[24]

The method for harvesting a culture product according to any of [1] to [23], wherein the filtration membrane permeation rate of the culture product at which the membrane area-based cumulative throughput of the filtration membrane is 400 L/m² is 70% or more of the filtration membrane permeation rate of the culture product at which the membrane area-based cumulative throughput of the filtration membrane is 50 L/m².

### Advantageous Effect of Invention

According to the present invention, more productive culture and harvesting of a culture product can be achieved in the culture of cells which produce the culture product.

### BRIEF DESCRIPTION OF THE DRAWING

FIG. 1 is a graph showing time-dependent change in the membrane area-based cumulative amount of proteins produced in the continuous culture in Example 1 (MF-SL(ATF)), Comparative Example 1 (MF-SL(TFF)), Comparative Example 2 (RT(Spectrum)(ATF)), and Comparative Example 3 (RT(Spectrum) (TFF)).

### DESCRIPTION OF EMBODIMENTS

Hereinafter, a mode for carrying out the present invention (hereinafter, referred to as the “present embodiment”) will be described in detail. The present invention is not intended to be limited by the embodiments given below, and various changes or modifications can be made in the present invention without departing from the spirit thereof.

The present embodiment relates to a method for harvesting a culture product contained in a culture solution in the culture of cells which produce the culture product.

In the present embodiment, the culture product produced by the cells is not particularly limited as long as the culture product is produced by the cells as a result of cell culture. Examples thereof include biologically active substances and more specifically include proteins, viruses, exosomes, and nucleic acids (miRNA, etc.). Particularly, a useful substance that can be used as a drug is preferred. Specific examples thereof include hormones, cytokines, growth factors, enzymes, plasma proteins, exosomes, virus-like particles, and immunoglobulins (antibodies). Such a useful substance can be obtained by the culture of cells which produce the useful substance. Preferably, the useful substance is biosynthesized by cells which produce the useful substance and released into the culture solution.

In the present embodiment, the cells which produce the culture product refer to cells which produce the desired culture product by use of intracellular protein synthesis reaction. Specific examples thereof include E. coli and CHO cells. For example, the following cell lines deposited with ATCC can be used: CRL12444, CRL12445, and CRL10762 lines. Cells engineered to have such ability to produce the culture product may be used. Conditions and medium composition, etc., for producing the culture product by the culture of the cells are not particularly limited as long as the culture product can be produced by the method.

In the present embodiment, the culture method is not limited as long as the culture product is produced by the cells. Suspension culture is preferred from the viewpoint of easy scale-up, easy control at a large scale, etc. Alternatively, the culture may be carried out in any mode. Examples of the mode include continuous culture, fed-batch culture, and batch culture. In this context, a spinner flask or the like may be provided in order to add a stirring function. For example, a magnetic stirrer or an impeller on a shaft may be used as the stirring function.

In the present embodiment, the culture is preferably continuous culture from the viewpoint of further enhancing the productivity of the culture product. The continuous culture is a cell culture method which involves discharging a spent culture solution while supplying a fresh culture solution in order to efficiently produce the culture product. Particularly, for efficiently producing the culture product, the culture of the cells which produce the culture product is preferably long-term high-density culture which involves: discharging a spent culture solution from a culture vessel while supplying a fresh culture solution into the culture vessel; and maintaining the growing environment of the cells which produce the culture product in the culture vessel under the optimum conditions (see e.g., Japanese Patent Laid-Open No. 61-257181).

In this context, examples of the optimum conditions for the culture of the cells which produce the culture product include the appropriate control of a glucose concentration of the culture solution in a culture vessel. The glucose concentration of the culture solution in a culture vessel can be controlled, for example, by taking a given amount of the culture solution out of the culture vessel, measuring the glucose concentration, and adjusting the amount of the fresh culture solution supplied or the amount of the spent culture solution discharged. Another example of the optimum conditions for the culture includes the appropriate control of a metabolite (lactic acid, etc.) level in the culture solution. In this case as well, the metabolite (lactic acid, etc.) level can be controlled by taking a given amount of the culture solution out of the culture vessel, measuring the metabolite level, and making adjustment in the same way as above.

The method for harvesting a culture product according to the present embodiment comprises, for example, the following steps:

A: culturing the cells which produce the culture product in the culture solution to produce the culture product;

B: sending the culture solution to a filtration membrane;

C: filtering the culture solution by alternating tangential flow filtration while changing the flow of the culture solution so as to cause a reciprocating motion thereof in a direction parallel with the surface of the filtration membrane to obtain a filtrate;

D: sending back a culture solution residue that has remained without permeating the filtration membrane; and

G: harvesting the culture product from the filtrate.

In the method for harvesting a culture product according to the present embodiment, each of these steps does not necessarily have to be carried out in the order presented and can be carried out such that long-term high-density culture can be achieved while the growing environment of the cells which produce the culture product in a culture vessel is maintained under the optimum conditions. For example, the steps B to D and G can be allowed to proceed while continuously performing the step A.

The method for harvesting a culture product according to the present embodiment may further comprise steps other than the aforementioned steps. For example, the method for harvesting a culture product according to the present embodiment may further comprise, simultaneously with any of the steps B to D or prior to or after any of the steps B to D,

E: continuously and/or intermittently supplying a fresh culture solution.

The method for harvesting a culture product according to the present embodiment may further comprise, after the step D,

F: discharging the culture solution residue that has remained without being filtered.

The method for harvesting a culture product according to the present embodiment can be carried out using a filtration apparatus equipped with a filtration membrane, and a culture vessel. The culture vessel can be provided with: an outlet through which the culture solution is sent to the filtration membrane; and an inlet through which the culture solution residue that has remained without permeating the filtration membrane is sent back. The outlet and the inlet may be the same or different. The culture vessel can be further provided with: an outlet through which the culture solution in the culture vessel is sampled; an inlet through which a fresh medium is supplied; and a discharge port through which the culture solution residue is discharged from the system. The culture vessel can be further provided with an inlet through which the filtrate that has passed through the filtration membrane is sent back to the culture vessel, as an apparatus for use in testing, etc.

Also, the filtration apparatus can be provided with: an inlet through which the culture solution from the culture vessel is sent to the filtration membrane; and an outlet through which the culture solution residue that has remained without permeating the filtration membrane is sent back to the culture vessel. The outlet and the inlet may be the same or different. The culture solution residue that has remained without permeating the filtration membrane may be sent back to the culture solution (culture vessel) that is introduced to the step of culturing the cells which produce the culture product in the culture solution to produce the culture product (step A) or the step of sending the culture solution to a filtration membrane (step B). The filtration apparatus can be further provided with: an outlet for a filtrate containing the culture solution and the produced culture product, wherein the filtrate has passed through the filtration membrane by alternating tangential flow filtration; and an outlet through which the culture solution residue that has remained without being filtered through the filtration membrane is discharged from the system.

The culture vessel and the filtration apparatus are appropriately connected to each other, if necessary via a solution sending unit. In the case of, for example, the continuous culture of the cells, the culture vessel and the filtration apparatus can be connected to each other via: an outlet through which the culture solution is sent from the culture vessel to the filtration membrane; and an inlet (different from the outlet) through which the culture solution residue that has remained without permeating the filtration membrane is sent back to the culture vessel. For the continuous culture, a pressure gauge and a weight scale for monitoring, various pumps (diaphragm pump, etc.), and the like can be appropriately disposed therein. The filtration apparatus is preferably a cylindrical container because the cylindrical container is suitable for alternating tangential flow filtration.

The culture product can be harvested from the filtrate after the filtration by a method known to those skilled in the art according to the type of each culture product. For example, the culture product contained in the filtrate may be harvested in this state of the solution or may be appropriately harvested by centrifugation, concentration, purification, or the like.

In the culture of the cells which produce the culture product according to the present embodiment, the culture solution is filtered by alternating tangential flow filtration which involves filtering the culture solution while changing the flow of the culture solution so as to cause a reciprocating motion thereof in a direction parallel with the surface of the filtration membrane, in order to achieve culture with the high productivity of the culture product. The alternating tangential flow filtration can be carried out using an apparatus for alternating tangential flow filtration, for example, ATF manufactured by Refine Technology, LLC, in combination with a filtration module having the filtration membrane.

The method of the present embodiment is suitably used for culturing the cells which produce the culture product, i.e., filtering the culture solution while continuously culturing the cells which produce the culture product. The method of the present invention may be carried out after performing the culture for a given period, for example, in the culture solution retained beforehand in a culture vessel so that the desired amount of the culture product is produced. In this respect, the culture may be continued during the filtration.

In this case, the method for harvesting a culture product according to the present embodiment comprises the steps B to D.

Also, this operation can further comprise the step E. In the step E, the filtration can be terminated by decreasing the amount of a fresh culture solution to be supplied.

In the present embodiment, a porous membrane substantially having no dense layer in the surface of the culture solution side is preferably used as the filtration membrane from the viewpoint of enhancing the productivity of the culture product. Specifically, according to a method for measuring an internal-surface pore size described in Examples mentioned later, 100 or more pores are observed under a microscope as to approximately 10 sites that do not overlap with each other and are not biased to a particular location on the membrane surface. The pores in the obtained microscope photographs are processed by circle approximation. The diameters of the 100 or more pores are determined from the areas thereof. In this operation, the presence or absence of the dense layer can be confirmed from the ratio of pores having a diameter smaller than 20 μm. The ratio of pores having a diameter smaller than 20 μm in the surface of the culture solution side is preferably 50% or less, more preferably 40% or less, even more preferably 30% or less, further preferably 20% or less, particularly preferably 10% or less, of all pores in the surface of the culture solution side from the viewpoint of using a porous membrane having no dense layer.

In the present embodiment, a porous membrane having an average pore size of 20 μm or larger and 100 m or smaller for the pores in the surface of the culture solution side is preferably used as the filtration membrane from the viewpoint of enhancing the productivity of the culture product. The average pore size of the filtration membrane can be confirmed by use of, for example, a method described in Examples mentioned later. During the alternating tangential flow filtration process, the retention and removal of hydrophobic substances, etc. are constantly repeated for membrane pores in the surface of the culture solution side of the filtration membrane. This can prevent a forming of a high-concentration layer due to the accumulation of a certain substance and can thus prevent the permeation rate of the certain substance, i.e., the target protein, from being reduced due to reduction in permeability caused by the high-concentration layer. For example, the filtration membrane having an average pore size of 20 μm or larger for the pores in the surface of the culture solution side can prevent the membrane pores from being clogged due to the sedimentation of the certain substance in the membrane surface. The filtration membrane having an average pore size of 100 μm or smaller can keep the strength of the filtration membrane within a reasonable range. The average pore size for the pores in the surface of the culture solution side is preferably 20 μm or larger and 100 μm or smaller, more preferably 30 μm or larger and 60 μm or smaller.

The porous membrane is preferably in the form of a hollow fiber membrane. The porous hollow fiber membrane is suitable for the filtration process in continuous culture or the like because this membrane permits filtration at a low pressure and causes little damage to the cells which produce the culture product. Particularly, high productivity can be achieved by using a porous hollow fiber membrane that is less likely to reduce the permeation rate of the protein in the course of clogging of the membrane in the filtration process and can maintain the permeation rate of the protein for a long period.

In the present embodiment, the filtration membrane is preferably constituted by a blend of a hydrophobic polymer and a hydrophilic polymer. Particularly, the hydrophilic polymer is preferably polyvinylpyrrolidone. The filtration membrane is preferably a porous hollow fiber membrane whose tube wall is constituted by a blend of a hydrophobic polymer and polyvinylpyrrolidone as a hydrophilic polymer. Use of the hydrophobic polymer in the porous hollow fiber membrane is preferred because the hydrophobic polymer can impart moderate mechanical strength thereto and can impart thereto durability that allows the membrane to resist that long-term use as in continuous culture or the like. In addition, the tube wall constituted by a blend containing a reasonable amount of polyvinylpyrrolidone as the hydrophilic polymer can prevent the membrane from being contaminated by the adsorption of hydrophobic substance particles derived from disrupted cells, antibodies, etc., and can prevent the recovery rate of the culture product from being reduced in purification steps for various drugs.

The polyvinylpyrrolidone content of the filtration membrane is preferably 0.2% by mass or more and 3% by mass or less based on the total mass of the porous hollow fiber membrane. Polyvinylpyrrolidone contained at 0.2% by mass or more can prevent the membrane pores being clogged due to contamination caused by the adsorption of hydrophobic substances, etc. Polyvinylpyrrolidone contained at 3% by mass or less can secure mechanical strength and can prevent the membrane pores from being clogged due to the swelling of the hydrophilic polymer. This can prevent increase in filtration resistance.

The filtration membrane used is preferably a membrane in which, when the tube wall is equally divided into three areas in the membrane thickness direction, the content ratio of polyvinylpyrrolidone in an outer peripheral area including an external surface which is the surface of the filtrate side is larger than the content ratio of polyvinylpyrrolidone in an inner peripheral area including an internal surface which is the surface of the culture solution side. This is because the membrane that retains particles (e.g., hydrophobic substance particles) smaller than the membrane pores in the inner peripheral area can exert a depth filtration effect during the filtration process, whereas this membrane can prevent the membrane pores in the outer peripheral area from being clogged due to the adsorption of the hydrophobic substance particles.

In the present embodiment, the filtration membrane is preferably a porous hollow fiber membrane in which the average pore size of the surface of the culture solution side is larger than the average pore size of the surface of the filtrate side, from the viewpoint of enhancing the productivity of the culture product. This is because the membrane has a depth filtration effect on the pores in the surface of the culture solution side by retaining hydrophobic substances or the like within the membrane pores, whereas this membrane has an effect of fractionating hydrophobic substances or the like during filtration on the surface of the filtrate side.

The weight-average molecular weight of polyvinylpyrrolidone contained in the filtration membrane is preferably 400000 or larger and 800000 or smaller from the viewpoint of attaining a solution viscosity suitable for the manufacture of the porous hollow fiber membrane.

The filtration membrane preferably has at least 50% or more, more preferably 60% or more, even more preferably 70% or more, particularly preferably 80% or more, of pores having a pore size of 20 μm or larger. The filtration membrane for use in continuous culture or the like requires long-term use and a high permeation throughput. The filtration membrane having 50% or more pores with a pore size of 50 μm or larger can retain, within the membrane, substances to be removed and can sufficiently provide a depth filtration effect.

The filtration membrane is preferably a porous membrane having a minimum pore size of 0.1 μm or larger and smaller than 1 μm. This porous membrane is preferably a hollow fiber membrane. The filtration membrane preferably has a layer having a pore size of 0.1 μm or larger and smaller than 1 am in the surface of the filtrate side. The pore size in the surface of the filtrate side is preferably 0.1 μm or larger and smaller than 1 μm. The pore size of 0.1 μm or larger can prevent the cells from being damaged by filtration resistance or a rise in pressure necessary for the filtration. The pore size of 1 μm or smaller can produce sufficient fractionation properties. The minimum pore size is more preferably 0.2 μm or larger and smaller than 0.8 μm, even more preferably 0.3 μm or larger and smaller than 0.6 μm.

The filtration membrane is preferably a porous hollow fiber membrane in which the membrane thickness of the tube wall is preferably 300 μm or larger and 1000 μm or smaller, more preferably 350 μm or larger and 800 μm or smaller. The membrane thickness can be measured by, for example, a method described in Examples mentioned later. The filtration membrane having a membrane thickness of 300 μm or larger can retain, within the membrane, substances to be removed and can sufficiently produce a depth filtration effect. Moreover, this filtration membrane can maintain an appropriate filtration rate. The filtration membrane having a membrane thickness of 1000 μm or smaller maintains an effective cross section area per module and can be excellent in filtration performance.

Preferably, the filtration membrane satisfies the following equation (I):

Cₒᵤₜ/Cᵢₙ≧2 (I)

wherein Cₒᵤₜ represents the content ratio of polyvinylpyrrolidone in the outer peripheral area, and
Cᵢₙ represents the content ratio of polyvinylpyrrolidone in the inner peripheral area.

The porous hollow fiber membrane that exhibits such a hydrophilic polymer distribution has a better depth filtration effect of the inner peripheral area and a better effect of preventing the membrane pores in the outer peripheral area from being clogged due to the adsorption of substances to be removed.

The filtration membrane is preferably a porous hollow fiber membrane having an inside diameter of 1000 μm or larger and 2000 μm or smaller. The culture solution becomes a high-density cell suspension in continuous culture or the like. The inside diameter of 1000 μm or larger can prevent the entrance of the hollow fibers from being clogged by aggregated suspended substances. The filtration membrane having an inside diameter of 2000 μm or smaller maintains an effective cross section per module and can be excellent in filtration performance.

In the present embodiment, for the filtration membrane containing a hydrophobic polymer, the hydrophobic polymer preferably comprises polysulfone. This hydrophobic polymer allows the porous hollow fiber membrane to have better strength against change in temperature or change in pressure and to exhibit high filtration performance.

In the present embodiment, the steps B to D can be carried out under the optimum conditions, thereby harvesting the culture product with high efficiency and maintaining the ratio of aggregates in the culture solution at a lower rate for a long period.

The step B is preferably carried out, for example, by sending a culture solution having cells density of 10×10⁵ cells/mL or higher and 2000×10⁵ cells/mL or lower from the culture vessel to the filtration membrane. At a low cell density of lower than 10×10⁵ cells/mL, the culture product of interest may be produced in a small amount. On the other hand, at a high cell density of higher than 2000×10⁵ cells/mL, the culture solution is in short supply of nutrient components, which may require replacing the medium soon.

The time interval between the sending of the culture solution in the step B and the sending back of the culture solution residue in the step D is preferably set to 3 seconds or longer and 26 seconds or shorter. This sending and sending back are typically performed using a pump (diaphragm pump, etc.). At a time interval of shorter than 3 seconds, the pump for sending may not secure stably supply due to limitations such as a lower limit to the operation of an apparatus. At a time interval of longer than 26 seconds, the cells in the culture solution reside for a longer time in the hollow fiber membrane and may therefore be damaged.

The flow volume for sending the culture solution in the step B and for sending back the culture solution residue in the step D is preferably set to 2 L/min·m² or larger and 40 L/min·m² or smaller. At a flow volume of smaller than 2 L/min·m², the cells in the culture solution reside for a longer time in the hollow fiber membrane and may therefore be damaged. At a flow volume of larger than 40 L/min·m², the pump may not secure stable supply.

In the case of culturing the cells by continuous culture, the flow rate for circulating the culture solution is preferably set to 2 L/min·m² or larger and 40 L/min·m² or smaller. At a flow rate of smaller than 2 L/min·m², the cells in the culture solution reside for a longer time in the hollow fiber membrane and may therefore be damaged. At a flow rate of larger than 40 L/min·m², the pump may not secure stable supply.

In the case of culturing the cells by continuous culture, the culture solution is preferably supplied in the step E at 10 L/day·m² or larger and 200 L/day·m² or smaller. At smaller than 10 L/day·m², sufficient nutrition may not be supplied to the cells. At larger than 200 L/day·m², the culture product concentration may not be sufficiently raised.

When the culture is performed, a glucose concentration in the culture solution is preferably controlled at 1 g/L or higher and 15 g/L or lower. The glucose concentration can be controlled by an approach known in the art, such as the adjustment of the amount of the culture solution supplied, the addition of glucose to the culture solution, and the replacement of the culture solution. More preferably, the lower limit of the glucose concentration can be 1.3 g/L or higher, 2 g/L or higher, 3 g/L or higher, 4 g/L or higher, or 5 g/L or higher, and the upper limit thereof can be 14 g/L or lower, 13 g/L or lower, 12 g/L or lower, 11 g/L or lower, or 10 g/L or lower. At a glucose concentration of lower than 1 g/L in the culture solution, sufficient glucose may not be supplied, resulting in reduction in cell density or survival rate and insufficient enhancement in the productivity of the culture product. On the other hand, it is possible to keep the glucose concentration in the culture solution at a concentration higher than 15 g/L by a method such as the elevation of a glucose concentration in a culture solution component or increase in the rate of replacement of the culture solution. In the former method, however, osmotic pressure in the culture solution may be difficult to control. In the latter method, the excessive supply of glucose may adversely affect the growth of the cells and the production of the product. In addition, the amount of the medium supplied may outstrip the yield of the product, resulting in the insufficient elevation of the culture product concentration in the culture solution.

For the culture, a lactic acid concentration in the culture solution is desirably controlled at 2 g/L or lower. The lactic acid concentration serves as an index that indicates the balance between glucose and oxygen supply and demand for the cells, and can be controlled by the adjustment of the amount of the culture solution supplied and the control of glucose and oxygen supply to the culture solution. At a lactic acid concentration exceeding 2 g/L in the culture solution due to excessive supply of glucose, oxygen shortage, etc., the cell growth or culture product concentration may not be sufficiently enhanced. The upper limit of the lactic acid concentration in the culture solution is more preferably 1.8 g/L or lower, 1.6 g/L or lower, 1.5 g/L or lower, or 1.4 g/L or lower, even more preferably 1.2 g/L or lower or 1.0 g/L or lower. The lower limit of the lactic acid concentration in the culture solution is not particularly limited and can be, for example, 0.3 g/L or higher.

In the case of continuous culture or the like, the ratio of the amount of the culture solution retained in the culture vessel to the area of the filtration membrane (culture solution amount/filtration membrane area ratio) is desirably 5 L/m² to 200 L/m². At a ratio of lower than 5 L/m², the liquid volume is too small with respect to the membrane area and may hinder circulation, etc. At a ratio of higher than 200 L/m², the liquid volume is too large with respect to the membrane area and may cause clogging.

In one aspect of the present embodiment, more productive culture (preferably continuous culture) that is less likely to reduce the filtration membrane permeation rate of the culture product can be achieved even when the culture is continuously performed. The filtration membrane permeation rate of the culture product 10 days after the start of the culture can be, for example, 70% or more of the filtration membrane permeation rate of the culture product 3 days after the start of the culture. When the culture product is, for example, immunoglobulin G (IgG), the filtration membrane permeation rate of IgG 10 days after the start of the culture may be 70% or more of the filtration membrane permeation rate of IgG 3 days after the start of the culture. The filtration membrane permeation rate of IgG 10 days after the start of the culture is preferably 75% or more, more preferably 80% or more, even more preferably 85% or more, particularly preferably 90% or more, 95% or more, or 98% or more, of the filtration membrane permeation rate of IgG 3 days after the start of the culture.

The filtration membrane permeation rate of the culture product can be measured by using, for example, a method described in Examples mentioned later. In a preferred aspect, when the cell density of the culture solution is 10×10⁵ cells/mL or larger and 2000×10⁵ cells/mL or smaller and the flow volume for sending the culture solution is 2 L/min·m² or larger and 40 L/min·m² or smaller, the filtration membrane permeation rate of IgG 10 days after the start of the culture is 70% or more of the filtration membrane permeation rate of IgG 3 days after the start of the culture, for example, in the case of continuous culture in which one or more of conditions such as the type of the cells, the number of cells, the composition of the culture solution, and the flow rate during the continuous culture are set to a condition described in Example 1 mentioned later.

In one aspect of the present embodiment, more productive culture (preferably continuous culture) that has a low ratio of aggregates of the culture product (e.g., IgG) in the culture solution and/or in the filtrate can be achieved even when the culture is continuously performed. The ratio of aggregates in the culture solution 10 days after the start of the culture can be, for example, less than 150% of the ratio of aggregates in the culture solution 2 days after the start of the culture, and the ratio of aggregates in the filtrate 10 days after the start of the culture can be less than 150% of the ratio of aggregates in the filtrate 2 days after the start of the culture. In a preferred aspect, when the cell density of the culture solution is 10×10⁵ cells/mL or larger and 2000×10⁵ cells/mL or smaller and the flow volume for sending the culture solution is 2 L/min·m² or larger and 40 L/min·m² or smaller, the ratio of aggregates described above is achieved, for example, in the case of continuous culture in which one or more of conditions such as the type of the cells, the number of cells, the composition of the culture solution, and the flow rate during the continuous culture are set to a condition described in Example 1 mentioned later.

In one aspect of the present embodiment, culture in which the rate of increase in protein concentration in the culture solution is controlled within a given range can be achieved when the culture is continuously performed. In this culture, it is possible to perform a culture wherein, for example, when the protein concentration in the culture solution at the start of the filtration is defined as 1, the relative protein concentration in the culture solution at which the membrane area-based cumulative amount of the culture solution filtered is 400 L/m² does not exceed 5. The increase in the relative protein concentration leads to membrane clogging and may reduce the permeation rate of the target protein. Since proteins accumulated in the culture solution include unnecessary proteins such as waste in addition to the product of interest, increase in the amount of proteins in the culture solution may deteriorate the cell culture environment. Furthermore, such proteins increased in the culture solution may promote the foaming of the culture solution and may hinder the efficiency of oxygen supply and carbon dioxide discharge at the gas-liquid interface. Accordingly, the rate of increase in the relative protein concentration is preferably quintuple or less, more preferably quadruple or less, thrice or less, or twice or less, even more preferably 1.5 or less times.

In one aspect of the present embodiment, culture in which reduction in filtration membrane permeation rate is controlled within a given range can be achieved when the culture is continuously performed. In this culture, for example, when the filtration membrane permeation rate of the culture product at which the membrane area-based cumulative throughput of the filtration membrane is 50 L/m² is defined as 100%, the filtration membrane permeation rate of the culture product at which the membrane area-based cumulative throughput of the filtration membrane is 400 L/m² can be 70% or more. The reduction in the filtration membrane permeation rate of the culture product may lead to reduction in production efficiency and might further cause the degradation of the target substance. Accordingly, the aforementioned filtration membrane permeation rate is more preferably 75% or more, 80% or more, or 85% or more, particularly preferably 90% or more.

In the present embodiment, one example of the filtration membrane as described above is a filtration membrane used in Examples below. Alternatively, in the present embodiment, a filtration membrane described in International Publication No. WO 2010/035793 may be used. Each measurement method can also be conducted according to the description of International Publication No. WO 2010/035793.

### EXAMPLES

Hereinafter, the present embodiment will be described more specifically with reference to Examples and Comparative Examples. However, the present embodiment is not intended to be limited by Examples below. The measurement methods used in the present embodiment are as follows:

### (1) Measurement of Internal-Surface Pore Size, and Confirmation of Position of Minimum-Pore Size Layer and Presence or Absence of Dense Layer

The internal surface of a freeze-dried porous hollow fiber membrane was observed under an electron microscope (manufactured by KEYENCE Corp., VE-9800) at a magnification where 10 or more pores were observable per visual field. The 10 pores in the obtained microscope photographs were processed by circle approximation. The average of diameters determined from the areas thereof was used as the internal-surface pore size. The serial cross sections from the internal surface side toward the external surface side of a freeze-dried porous hollow fiber membrane were observed under a microscope to confirm the position of a layer having a minimum cross-sectional pore size (minimum-pore size layer).

The structure of the innermost surface of a porous hollow fiber membrane was observed under a microscope to confirm the presence or absence of a dense layer, based on the internal-surface pore size. Specifically, 10 pores were observed per visual field as mentioned above to observe 100 or more pores in approximately 10 visual fields that did not overlap with each other and were not biased to a particular location on the membrane surface. When the ratio of pores having a diameter smaller than 20 μm exceeded 50%, the dense layer was confirmed to be present. When the ratio of pores having a diameter smaller than 20 μm was 50% or less, the dense layer was confirmed to be absent.

### (2) Method for Determining Pore Size of Minimum-Pore Size Layer

Polystyrene latex particles (manufactured by JSR Corp., SIZE STANDARD PARTICLES) were dispersed in an aqueous solution containing 0.5% by mass of sodium dodecyl sulfate (manufactured by Wako Pure Chemical Industries, Ltd.) such that the particle concentration was 0.01% by mass to prepare a latex particle dispersion.

The latex particle dispersion was filtered using a porous hollow fiber membrane. Change in the concentration of the latex particles between before and after the filtration was measured. This measurement was conducted with the latex particle size changed from 0.1 μm at an interval of approximately 0.1 μm to prepare an inhibition curve of the latex particles. From this inhibition curve, a particle size at which the permeation of 98% particles can be inhibited was read. This diameter was used as the pore size of the minimum-pore size layer (inhibition pore size).

When the minimum-pore size layer can be confirmed to be present in the outer peripheral area according to “(1) Confirmation of position of minimum-pore size layer”, the pore size of the minimum-pore size layer (inhibition pore size) determined according to “(2) Method for determining pore size of minimum-pore size layer” is the inhibition pore size of the outer peripheral area.

### (3) Measurement of Inside Diameter, Outside Diameter, and Membrane Thickness of Porous Hollow Fiber Membrane

A porous hollow fiber membrane was sliced into a circular tube form, which was observed under an optical microscope (manufactured by KEYENCE Corp., VH6100) to measure the inside diameter (μm) and the outside diameter (μm) of the porous hollow fiber membrane. From the obtained inside diameter and outside diameter, the membrane thickness was calculated according to the following equation (II):

Membrane thickness (μm)=(Outside diameter−Inside diameter)/2 (II)

### (4) Measurement of Protein Concentration and Permeation Rate

The protein concentrations of a filtrate after filtration and a culture solution in a culture vessel at the time of continuous culture were quantitatively analyzed by ELISA.

The permeation rate of the protein for a porous hollow fiber membrane was calculated according to the following equation (III):

Permeation rate X of the protein=(Protein concentration of the filtrate)/(Protein concentration of the culture solution in a culture vessel when the filtrate was sampled)×100 (III)

### (5) Measurement of Total Cell Density and Cell Survival Rate in Culture Vessel

A culture solution in a culture vessel during continuous culture was sampled, and the total cell density and the cell survival rate of the sample were measured by using an automatic cell count apparatus (manufactured by GE Healthcare Japan Corp., CYTORECON).

### (6) Measurement of Ratio of IgG Aggregates in Culture Solution and Filtrate

A commercially available affinity chromatography media-packed column (MabSelect, GE Healthcare Japan Corp.) was used in the purification of IgG from a culture solution or a filtrate. Antibody adsorption and elution conditions abided by the instruction attached to the product. A solution for eluting the antibody from the media-packed column had a hydrogen ion exponent of pH 3.0. The hydrogen ion exponent of the harvested eluate was set to pH 5.0 by titration using a 1 mol/L tris-HCl buffer solution (pH 8.0).

The ratio of antibody aggregates in the obtained antibody preparation was measured by using a size-exclusion high-performance liquid chromatography system. Specifically, the system in which a reservoir tank (mobile phase: 0.1 mol/L phosphoric acid and 0.2 mol/L arginine, pH 6.8), a solution sending pump (linear velocity of solution sending: 1.68 cm/min), a sample loop (volume: 100 μL), a column (room temperature), a detector (UV, wavelength: 280 nm), and a drain were connected in the order presented was used to load the antibody preparation. Then, the ratio of aggregates contained in the antibody preparation was quantified from absorbance detected in the detector. Tosoh TSKGEL G3000SWXL column having an inside diameter (diameter) of 7.8 mm and a bed height of 300 mm was used. Typically, the peak of dimers or larger aggregates (peak A) is detected before an elution time of 16 minutes, while the monomer peak (peak B) was detected at an elution time of 16 minutes to 18 minutes. From the areas of these peaks, the ratio of antibody aggregates was calculated according to the following equation (IV):

Ratio of aggregates (%)=100×(Area of peak A)/(Area of peak A+Area of peak B) (IV)

### (7) Measurement of Glucose Concentration and Lactic Acid Concentration

The glucose concentration and the lactic acid concentration in a culture solution were measured by using a 4-channel biosensor BF-6M (Oji Scientific Instruments). A method, reagents, and consumable goods all abided by the manual attached to the instrument. Specifically, the culture solution was diluted 2-fold with saline and assayed by using an autosampler BF-30AS (Oji Scientific Instruments). The electrodes used in the glucose measurement and the lactic acid measurement were a glucose electrode #ED05-0003 for planar replacement and a novel L-lactic acid electrode #ED05-0001 for planar replacement, respectively, manufactured by Oji Scientific Instruments. The buffer solution, washing solution, and standard solutions used were a buffer solution (for BF) #SL03-0002, a washing solution (for AS) #SL01-0001, and standard solution/glucose 0.54% #SL23-0003 and standard solution/L-lactic acid 5 mM #SL21-0013, respectively, manufactured by Oji Scientific Instruments.

### Example 1

Chinese hamster ovary (CHO) cells were cultured in a serum-free medium (Invitrogen Corp., CD opti CHO AGT without 2ME) to obtain a CHO cell suspension.

A 12-L cell culture vessel, a porous hollow fiber membrane module (minimodule prepared using a hollow fiber membrane installed in BioOptimal MF-SL manufactured by Asahi Kasei Medical Co., Ltd., membrane area: 0.085 m²) as a membrane for separating the cells in the cell culture solution from the spent medium, an alternating tangential flow filtration system for continuous culture (ATF-2, manufactured by Refine Technology, LLC) loaded with the membrane, and a medium tank for supplying an unused medium to the culture vessel were all connected in advance and sterilized by autoclaving. The culture vessel was provided with: an outlet through which the culture solution was sent from the culture vessel to the porous hollow fiber membrane; an outlet through which the culture solution in the culture vessel was sampled; and an inlet through which a fresh medium was supplied.

To 4.5 L of a fresh serum-free medium, human immunoglobulin G (manufactured by Japan Blood Products Organization, Venoglobulin IH 5% I.V. 2.5 g/50 mL) was added at a concentration of 0.5 mg/mL with respect to the culture solution. This mock culture solution was injected to the culture vessel. Further, 1 L of the CHO cell suspension having a cell density of 5×10⁵ cells/mL was injected thereto to start culture. Then, after confirmation of cell growth into 1.5×10⁷ cells/mL as the total number of cells, the liquid volume in the culture vessel was adjusted to 4 L by the removal of an aliquot of the cell culture solution. Then, the alternating tangential flow filtration system was actuated to start the filtration of the culture solution and continuous culture in which an unused medium containing human immunoglobulin G added at a concentration of 0.5 mg/mL with respect to the culture solution was supplied as a mock culture solution to the culture vessel.

The culture solution was sent from the culture vessel to the porous hollow fiber membrane by using a diaphragm pump in the alternating tangential flow filtration system, to perform filtration. The amount of the solution sent was set to 0.5 to 1.2 L/min (6 to 14 L/min·m²) such that the amount of the solution sent was 250 times the amount of the permeate. The rate of medium replacement was set to 1 [total amount of the culture solution/day] at the start of the continuous culture and increased to within a range of 0.75 to 1.75 [total amount of the culture solution/day] (=approximately 35 to approximately 82 L/day·m²) according to increase in the total cell density in the culture vessel. The filtrate outlet of the porous hollow fiber membrane was provided with: a pump through which the filtrate was extracted at the same given rate as in the amount of the unused medium supplied; and a weight scale that permitted measurement of the amount of the filtrate at any time.

The temperature and the oxygen and air supply were controlled using a cell culture apparatus #BJR-S10NA1S-8C (manufactured by ABLE Co., Ltd.). The amount of dissolved oxygen (DO) was measured using an oxygen sensor Inpro 6800/12/420 (manufactured by Mettler-Toledo International Inc.) connected to this apparatus.

The oxygen and air supply was controlled such that only air was supplied at a DO level of 6 ppm or higher, and oxygen was also supplied at a DO level of below 6 ppm. Throughout the culture, the glucose concentration and the lactic acid concentration were controlled within ranges of 1.3 to 10 g/L and 0.7 to 1.5 g/L, respectively. The glucose concentration was controlled by the adjustment of the rate of medium replacement. The lactic acid concentration was controlled by the adjustment of the rate of medium replacement.

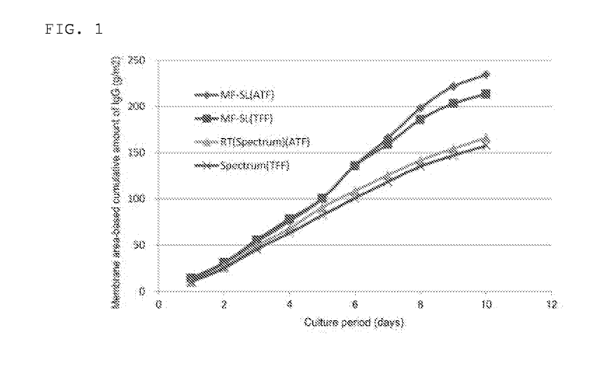

The culture vessel and the filtrate were sampled once a day, and the total cell density, the cell survival rate, and the protein concentration were measured. From the protein concentration, the permeation rate of the protein was calculated. From the protein concentration of the filtrate, the weight of the filtrate, and the m²-based cumulative amount of the protein produced converted using the membrane area of the porous hollow fiber membrane, was calculated. The results are shown in Table 1 and FIG. 1.

At culture day 10, the total cell density was 5.30×10⁷ cells/mL; the cell survival rate was 76.47%; the permeation rate of the protein was 82.1%; and the cumulative amount of the protein produced was 234.2 g/m².

The cell survival rate, the permeation rate of the protein, and the cumulative amount of the protein produced were higher than those in Comparative Examples 1 to 3, demonstrating the usefulness of the method of the present invention.

### Comparative Example 1

A 12-L cell culture vessel, a porous hollow fiber membrane module (minimodule prepared using a hollow fiber membrane installed in BioOptimal MF-SL manufactured by Asahi Kasei Medical Co., Ltd., membrane area: 0.085 m²) as a membrane for separating the cells in the cell culture solution from the spent medium, and a medium tank for supplying an unused medium to the culture vessel were all connected in advance and sterilized by autoclaving. The culture vessel was provided with: an outlet through which the culture solution was sent from the culture vessel to the porous hollow fiber membrane; an inlet through which the solution that passed through the porous hollow fiber membrane was sent back to the culture vessel; an outlet through which the culture solution in the culture vessel was sampled; and an inlet through which a fresh medium was supplied.

To 4.5 L of a fresh serum-free medium, human immunoglobulin G was added at a concentration of 0.5 mg/mL with respect to the culture solution. This mock culture solution was injected to the culture vessel. Further, 1 L of the CHO cell suspension having a cell density of 5×10⁵ cells/mL was injected thereto to start culture.

Then, after confirmation of cell growth into 1.5×10⁷ cells/mL as the total number of cells, the liquid volume in the culture vessel was adjusted to 4 L by the removal of an aliquot of the cell culture solution. Then, filtrate was started. The filtration was carried out by tangential flow filtration using a peristaltic pump to send the solution from the culture vessel to the porous hollow fiber membrane. For continuous culture, the amount of the solution sent was set such that the shear rate was 2900 s⁻¹. An unused medium containing human immunoglobulin G added at a concentration of 0.5 mg/mL with respect to the culture solution was supplied as a mock culture solution within a range of 1 to 1.75 [total amount of the culture solution/day] to the culture vessel. The filtrate outlet of the porous hollow fiber membrane was provided with: a pump through which the filtrate was extracted at the same given rate as in the amount of the unused medium supplied; and a weight scale that permitted measurement of the amount of the filtrate at any time.

Sampling was carried out in the same way as in Example 1. Results of the measurement of various items are shown in Table 1. At culture day 10, the total cell density was 4.32×10⁷ cells/mL; the cell survival rate was 75%; the permeation rate of the protein was 68%; and the cumulative amount of the protein produced was 213.5 g/m².

### Comparative Example 2

Continuous culture was carried out using the alternating tangential flow filtration system in the same way as in Example 1 except that: MF hollow fiber module manufactured by Refine Technology, LLC (inhibition pore size: 0.2 m, membrane area: 0.13 m²) was used as the porous hollow fiber membrane; and the membrane area-based liquid volume was adjusted to the same level as in Example 1.

Sampling was carried out in the same way as in Example 1. Results of the measurement of various items are shown in Table 1. At culture day 10, the total cell density was 4.39×10⁷ cells/mL; the cell survival rate was 73%; the permeation rate of the protein was 41%; and the cumulative amount of the protein produced was 166.1 g/m².

### Comparative Example 3

Continuous culture was carried out in the same way as in Comparative Example 1 except that: Midicross X32E-301-02N manufactured by Spectrum Laboratories, Inc. (inhibition pore size: 0.2 μm, membrane area: 0.0065 m²) was used as the porous hollow fiber membrane; and the membrane area-based liquid volume was adjusted to the same level as in Example 1.

Sampling was carried out in the same way as in Example 1. Results of the measurement of various items are shown in Table 1. At culture day 10, the total cell density was 3.73×10⁷ cells/mL; the cell survival rate was 68%; the permeation rate of the protein was 38%; and the cumulative amount of the protein produced was 157.6 g/m².

TABLE 1

| | Example. 1 | | | | Comparative Example. 1 | | | |
|---|---|---|---|---|---|---|---|---|
| Porous hollow | BioOptimal ™ MF-SL | | | | BioOptimal ™ MF-SL | | | |
| fiber membrane | | | | | | | | |
| Internal-surface | 30-80 | | | | 30-80 | | | |
| pore size (μm) | | | | | | | | |
| Innermost- | None | | | | None | | | |
| surface dense | | | | | | | | |
| layer | | | | | | | | |
| Inhibition pore | 0.40 | | | | 0.40 | | | |
| size (μm) | | | | | | | | |
| Inside diameter | 1.4 | | | | 1.4 | | | |
| (mm) | | | | | | | | |
| Outside | 2.3 | | | | 2.3 | | | |
| diameter (mm) | | | | | | | | |
| Membrane | 0.45 | | | | 0.45 | | | |
| thickness (mm) | | | | | | | | |
| Culture solution | ATF | | | | TTF | | | |
| circulation | | | | | | | | |
| method | | | | | | | | |
| The number of | The number | Survival | Permeation | Cumulative | The number | Survival | Permeation | Cumulative |
| culture days | of cells | rate | rate of | amount of | of cells | rate | rate of | amount of |
| (days) | (cells/mL) | (%) | IgG (%) | IgG (g/m2) | (cells/mL) | (%) | IgG (%) | IgG (g/m2) |
| 3 | 358 × 10⁵ | 93.12 | 100 | 53.3 | 318 × 10⁵ | 89.36 | 100 | 55.5 |
| 8 | 596 × 10⁵ | 82.23 | 97.4 | 198.2 | 420 × 10⁵ | 83 | 79 | 185.8 |
| 10 | 532 × 10⁵ | 76.47 | 82.1 | 234.2 | 432 × 10⁵ | 75 | 68 | 213.5 |
| | Comparative Example. 2 | | | | Comparative Example. 3 | | | |
| Porous hollow | MF hollow fiber module manufactured | | | | Midicross X32E-301-02N manufactured | | | |
| fiber membrane | by Refine Technology, LLC (Spectrum) | | | | by Spectrum Laboratories, Inc. | | | |
| Internal-surface | 2-4 | | | | 2-4 | | | |
| pore size (μm) | | | | | | | | |
| Innermost- | Present | | | | Present | | | |
| surface dense | | | | | | | | |
| layer | | | | | | | | |
| Inhibition pore | 0.20 | | | | 0.20 | | | |
| size (μm)) | | | | | | | | |
| Inside diameter | 1.0 | | | | 1.0 | | | |
| (mm) | | | | | | | | |
| Outside | 1.3 | | | | 1.3 | | | |
| diameter (mm) | | | | | | | | |
| Membrane | 0.15 | | | | 0.15 | | | |
| thickness (mm) | | | | | | | | |
| Culture solution | ATF | | | | TFF | | | |
| circulation | | | | | | | | |
| method | | | | | | | | |
| The number of | The number | Survival | Permeation | Cumulative | The number | Survival | Permeation | Cumulative |
| culture days | of cells | rate | rate of | amount of | of cells | rate | rate of | amount of |
| (days) | (cells/mL) | (%) | IgG(%) | IgG(g/m2) | (cells/mL) | (%) | IgG(%) | IgG(g/m2) |
| 3 | 295 × 10⁵ | 91.45 | 91.9 | 48.7 | 318 × 10⁵ | 89.36 | 78 | 45.3 |
| 8 | 470 × 10⁵ | 77.3 | 56 | 141.6 | 370 × 10⁵ | 77 | 44 | 135.1 |
| 10 | 439 × 10⁵ | 73 | 41 | 166.1 | 373 × 10⁵ | 68 | 38 | 157.6 |

Example 1 and Comparative Example 2 were compared in terms of protein concentration in the culture solution in the culture vessel and the permeation rate at the point in time when the throughput of the culture solution per m² of membrane area (membrane area-based cumulative throughput) reached 50 L or 400 L. The relative protein concentration at the start of the filtration was defined as 1. The permeation rate of the protein at which the membrane area-based cumulative throughput was 50 L/m² was defined as 100%. The relative protein concentration and the relative permeation rate of the protein at which the membrane area-based cumulative throughput was 400 L/m² are shown in Table 2.

TABLE 2

| Membrane | Example 1 | | Comparative Example 2 | |
|---|---|---|---|---|
| area-based | Relative | | | Relative |
| cumulative | protein | Relative | | permeation |
| throughput | concen- | permeation | Relative protein | rate of |
| (L/m²) | tration | rate of protein | concentration | protein |
| 0 | 1 | | 1 | |
| 50 | | 100% | | 100% |
| 400 | 1.5 | 97% | 2.1 | 48% |

At the point in time when the membrane area-based cumulative throughput reached 400 L/m², in Example 1, protein accumulation in the culture solution was suppressed and the relative permeation rate was kept high. On the other hand, in Comparative Example 2, increase in protein concentration in the culture solution and reduction in relative permeation rate were observed. The proteins accumulated in the culture solution may lead to further reduction in permeability. Since the proteins accumulated in the culture solution include proteins such as waste in addition to the product of interest, increase in the amount of proteins in the culture solution may deteriorate the cell culture environment. These results demonstrated that the method used in Example 1, compared with the method used in Comparative Example 2, can maintain an environment desirable for the growth of the cells and the production of the product and can achieve high productivity.

The hollow fiber membrane used in Example 1 and Comparative Example 1 was a hollow fiber membrane in which:

the hollow fiber membrane is constituted by a blend of polysulfone and polyvinylpyrrolidone;

the polyvinylpyrrolidone content is 1.2% by mass based on the total mass of the porous hollow fiber membrane;

the content ratio of polyvinylpyrrolidone in the outer peripheral area including the external surface (on the filtrate side) is larger than the content ratio of polyvinylpyrrolidone in the inner peripheral area including the internal surface (on the culture solution side);

the weight-average molecular weight of polyvinylpyrrolidone contained in the filtration membrane is a grade of 440,000;

the membrane has pores wherein 90% of the pores have a pore size of 20 μm or larger; and

the value of Cₒᵤₜ/Cᵢₙ [Cₒᵤₜ represents the content ratio of polyvinylpyrrolidone in the outer peripheral area, and Cᵢₙ represents the content ratio of polyvinylpyrrolidone in the inner peripheral area] is approximately 2.7.

The hollow fiber membrane used in Comparative Examples 2 and 3 was a hollow fiber membrane made of polyether sulfone.

Results of further comparing Example 1 with Comparative Examples 2 and 3 in terms of the ratio of product aggregates in the culture solution and the filtrate are shown below.

At the early stage of the culture (day 2), no difference was observed in the ratio of aggregates in the culture solution and the filtrate, depending on the use of different membrane module. At the later stage of the culture (day 10), increase in the ratio of aggregates was suppressed only in Example 1 (using BioOptimal MF-SL). These results demonstrated the usefulness of the method of the present invention in suppressing increase in the ratio of impurities when the culture is continuously performed.

TABLE 3

| | | Ratio of | | Ratio of | |
|---|---|---|---|---|---|
| | | aggregates in | | aggregates in | |
| | Porous hollow fiber | culture solution | | filtrate | |
| | membrane module | day 2 | day 10 | day 2 | day 10 |
| Example 1 | BioOptimal MF-SL | 3% | 3% | 3% | 3% |
| Comparative | MF hollow fiber module | 3% | 7% | 3% | 6% |
| Example 2 | manufactured by Refine | | | | |
| | Technology, LLC | | | | |
| | (manufactured | | | | |
| | by Spectrum | | | | |
| | Laboratories, Inc.) | | | | |
| Comparative | Midicross | 3% | 6% | 3% | 6% |
| Example 3 | X32E-301-02N | | | | |
| | manufactured | | | | |
| | by Spectrum | | | | |
| | Laboratories, Inc. | | | | |

### Example 2

Culture was carried out under conditions where: the medium supply rate during the culture was fixed to 0.75 [total amount of the culture solution/day](=approximately 35 L/day·m²) or 1.75 [total amount of the culture solution/day](=approximately 82 L/day·m²) after the start of the filtration; and the glucose concentration was not controlled. The other conditions were the same as in Example 1. In the former case, the glucose concentration was 1 g/L or lower on the next day of the start of the filtration, and the cell survival rate was 10% or less at filtration day 3. The membrane area-based cumulative amount of IgG (g/m²) was 65 at filtration day 3. In the latter case, the glucose concentration of 10 g/L was maintained up to filtration day 10, whereas the cell density remained at approximately 3×10⁷ cells/mL at the maximum and the membrane area-based cumulative amount of IgG (g/m²) was 165 at filtration day 10.

### Example 3

Culture was carried out under conditions where: only air was supplied without supplying oxygen after the start of the filtration; and the control of the lactic acid concentration at 2 g/L or lower was not carried out. The other conditions were the same as in Example 1. The DO value fell below 6 ppm at filtration day 2 and became 0 ppm at filtration day 4. The lactic acid concentration exceeded 2 g/L at filtration day 4 and then exhibited 2 g/L or larger up to filtration day 7. The cell survival rate was 10% or less at culture day 8. The membrane area-based cumulative amount of IgG (g/m²) was 68 at culture day 8.

The results of Examples 1, 2, and 3 demonstrated that the control of the glucose concentration and the lactic acid concentration within the particular ranges is useful in enhancing the productivity of the culture product by the cells.

### INDUSTRIAL APPLICABILITY

The present invention can provide a more productive method for culturing and a more productive method for harvesting culture product in cell culture wherein the cell produces the culture product and thus has industrial applicability. The present invention is useful in the fields of bio-pharmaceuticals, etc.

The present application claims the priority based on Japanese Patent Application No. 2014-183641 filed on Sep. 9, 2014, the content of which is incorporated herein by reference.

1. A method for harvesting a culture product contained in a culture solution in cell culture wherein the cell produces the culture product, comprising following steps:
B: sending the culture solution to a filtration membrane;
C: filtering the culture solution by alternating tangential flow filtration while changing the flow of the culture solution so as to cause a reciprocating motion thereof in a direction parallel with the surface of the filtration membrane to obtain a filtrate;
D: sending back a culture solution residue that has remained without permeating the filtration membrane; and
G: harvesting the culture product from the filtrate, wherein
the filtration membrane used in the step B is; a porous membrane having an average pore size of 20 μm or larger and 100 μm or smaller for the pores in the surface of the culture solution side: or a porous membrane in which the ratio of pores having a diameter smaller than 20 μm for the pores in the surface of the culture solution side is 50% or less of all pores in the surface of the culture solution side.

2. (canceled)

3. The method for harvesting a culture product according to claim 1, wherein the filtration membrane is a porous membrane in which the average pore size of the pores in the surface of the culture solution side is larger than the average pore size of the pores in the surface of the filtrate side.

4. The method for harvesting a culture product according to claim 1, wherein the filtration membrane is a hollow fiber membrane.

5. The method for harvesting a culture product according to claim 1, wherein the filtration membrane has a minimum pore size of 0.1 μm or larger and 1 μm or smaller.

6. The method for harvesting a culture product according to claim 1, wherein the filtration membrane is a porous hollow fiber membrane constituted by a blend of a hydrophobic polymer and polyvinylpyrrolidone.

7. The method for harvesting a culture product according to claim 6, wherein the filtration membrane is a hollow fiber membrane in which, when the tube wall is equally divided into three areas in the membrane thickness direction, the content ratio of polyvinylpyrrolidone in an outer peripheral area including an external surface which is the surface of the filtrate side of the filtration membrane is larger than the content ratio of polyvinylpyrrolidone in an inner peripheral area including an internal surface which is the surface of the culture solution side of the filtration membrane.

8. The method for harvesting a culture product according to claim 6, wherein the hydrophobic polymer is polysulfone.

9. The method for harvesting a culture product according to claim 1, further comprising, prior to the step B,
A: culturing the cell which produces the culture product in the culture solution to produce the culture product.

10. The method for harvesting a culture product according to claim 1, further comprising, simultaneously with any of the steps B to D or prior to or after any of the steps B to D,
E: continuously and/or intermittently supplying a fresh culture solution.

11. The method for harvesting a culture product according to claim 1, further comprising, after the step D,
F: discharging the culture solution residue.

12. The method for harvesting a culture product according to claim 9, wherein the step A is carried out in the culture solution retained in a culture vessel.

13. The method for harvesting a culture product according to claim 1, wherein the step C is carried out using the filtration membrane housed in a cylindrical container.

14. The method for harvesting a culture product according to claim 1, wherein the filtration membrane permeation rate of the culture product 10 days after the start of the culture is 70% or more of the filtration membrane permeation rate of the culture product 3 days after the start of the culture.

15. The method for harvesting a culture product according to claim 1, wherein the ratio of aggregates in the culture solution 10 days after the start of the culture is less than 150% of the ratio of aggregates in the culture solution 2 days after the start of the culture.

16. The method for harvesting a culture product according to claim 1, wherein the ratio of aggregates in the filtrate 10 days after the start of the culture is less than 150% of the ratio of aggregates in the filtrate 2 days after the start of the culture.

17. The method for harvesting a culture product according to claim 1, wherein the culture product is a biologically active substance.

18. The method for harvesting a culture product according to claim 1, wherein the culture product is selected from the group consisting of proteins, viruses, exosomes, and nucleic acids.

19. The method for harvesting a culture product according to claim 1, wherein the culture product is an immunoglobulin.

20. The method for harvesting a culture product according to claim 1, wherein the culture is continuous culture.

21. The method for harvesting a culture product according to claim 1, wherein a glucose concentration in the culture solution is controlled at 1 g/L or higher and 15 g/L or lower.

22. The method for harvesting a culture product according to claim 1, wherein a lactic acid concentration in the culture solution is controlled at 0 g/L or higher and 2 g/L or lower.

23. The method for harvesting a culture product according to claim 1, wherein the protein concentration of the culture solution at which the membrane area-based cumulative throughput of the filtration membrane is 400 L/m² is twice or less the protein concentration of the culture solution at the start of the filtration.

24. The method for harvesting a culture product according to claim 1, wherein the filtration membrane permeation rate of the culture product at which the membrane area-based cumulative throughput of the filtration membrane is 400 L/m² is 70% or more of the filtration membrane permeation rate of the culture product at which the membrane area-based cumulative throughput of the filtration membrane is 50 L/m².
